(19) United States
(12) Patent Application Publication
(10) Pub. No.: US 2015/0045705 A1
(43) Pub. Date: Feb. 12, 2015

(54) TEETH GRINDING PREVENTING DEVICE

(71) Applicant: Pacific Medico Co., Ltd., Tokyo (JP)

(72) Inventors: Kazuyoshi Baba, Tokyo (JP); Glenn T. Clark, Los Angeles, CA (US)

(21) Appl. No.: 14/444,389

(22) Filed: Jul. 28, 2014

(30) Foreign Application Priority Data
Aug. 9, 2013 (JP) 2013-166346

(51) Int. Cl. A61F 5/56; A61H 23/00; A61H 21/00; A61B 5/00

(52) U.S. Cl. CPC A61F 5/566; A61B 5/682; A61B 5/4836; A61B 5/4557; A61H 23/00; A61H 21/00; A61F 2005/563; A61H 2201/5061
USPC 601/46; 128/861

(57) ABSTRACT

A teeth grinding preventing device to be put in an oral cavity of a user includes a main body portion formed into a substantially arcuate shape along a curve of a maxillary teeth alignment of the user and configured to cover an occluding surface of the maxillary teeth, a sensor portion embedded in the main body portion to extend in parallel to the occluding surfaces of maxillary and mandibular teeth and configured to detect an occlusal force and output a detection signal, a teeth grinding determining portion configured to receive the detection signal and determine whether or not the user is grinding the teeth, and a stimulation providing portion configured to provide the user with a stimulation when the teeth grinding determining portion determines that the user is grinding the teeth, and the stimulation providing portion is provided to be mountable and demountable with respect to the main body portion.

### BACKGROUND OF THE INVENTION

1. Field of the Invention

The present invention relates to a teeth grinding preventing device configured to detect and prevent teeth grinding of a user.

2. Description of the Related Art

Teeth grinding during sleep causes abrasion of teeth by friction between teeth and teeth, and is considered to be a cause of temporomandibular disorder by an application of an excessive load on temporomandibular joint. Since teeth grinding generates uncomfortable sounds, sleep of persons sleeping in the same room is disturbed. In addition, since teeth grinding during sleep occurs on an unselfconscious level, an improvement is significantly difficult even though the person has a consciousness of stopping teeth grinding.

Teeth grinding therapy has been studied for many years. As an example, JP-A-10-234752 discloses a device, which is a teeth grinding-preventing mouthpiece to be formed by a specific dentist from a mold matching an individual denture model, and to be put into the mouth to prevent teeth grinding. JP-A-2004-329242 also discloses a teeth grinding preventing device which avoids necessity of manufacturing individual denture models.

However, the teeth grinding preventing devices disclosed in JP-A-10-234752 and JP-A-2004-329242 are configured to avoid friction between teeth and teeth to prevent abrasion of teeth and is not configured to resolve a symptom “teeth grinding” itself. In contrast, as one of therapies that restrain the symptom “teeth grinding”, there is a biofeedback therapy. The biofeedback therapy is a method of providing a sort of stimulation (sounds or contact stimulation) when the teeth grinding is detected, and is known as having an effect of resolving the teeth grinding itself. The biofeedback therapy on the basis of sounds is suspected to disturb sleep of a person being tested by generated sounds.

### SUMMARY OF THE INVENTION

In view of such problems described above, it is an object of the present invention to provide a teeth grinding preventing device.

A teeth grinding preventing device of the invention for solving the problem described above is a teeth grinding preventing device to be put in an oral cavity of a user including: a main body portion formed into a substantially arcuate shape along a curve of a maxillary teeth alignment of the user, and configured to cover an occluding surface of the maxillary teeth; a sensor portion embedded in the main body portion so as to extend in parallel to the occluding surface of maxillary and mandibular teeth, and configured to detect an occlusal force and output a detection signal; a teeth grinding determining portion configured to receive the detection signal and determine whether or not the user is grinding the teeth; and a stimulation providing portion configured to provide the user with a stimulation when the teeth grinding determining portion determines that the user is grinding the teeth, wherein the stimulation providing portion is provided so as to be mountable and demountable with respect to the main body portion.

In this configuration, when the teeth grinding is detected, a stimulation is applied to the user having the teeth grinding preventing device of the invention put in the oral cavity, so that a restraint of teeth grinding is encouraged.

The teeth grinding preventing device of the invention preferably includes a storage portion in which time-history data of the occlusal force is to be stored.

In this configuration, the time-history data of the occlusal force may be stored and may be utilized for an analysis of the teeth grinding or the like. The time-history data includes data of changes in occlusal force with elapse of time.

The teeth grinding preventing device of the invention further includes a transmitting portion configured to transmit the time-history data of the occlusal force stored in the storage portion to a user information communication terminal.

In this configuration, the time-history data of the occlusal force may be transmitted to the user information communication terminal owned by the user, for example, a smart phone, a tablet terminal, or a laptop personal computer owned by the user.

Further, in the teeth-grinding preventing device of the invention, the stimulation providing portion provides the user with a vibratory stimulation.

In this configuration, sleep of the user is not disturbed, and sleep of persons sleeping in the same room is not disturbed.

Further, in the teeth-grinding preventing device of the invention, the stimulation providing portion is provided so as to protrude from a top of the main body portion out of the oral cavity.

In this configuration, the portion to be arranged in the oral cavity of the user can be reduced, so that a cause which gives a sense of discomfort to the user is reduced.

Further, in the teeth-grinding preventing device of the invention, the sensor portion is composed of a piezoelectric element embedded in a substantially arcuate shape.

In this configuration, the teeth grinding of the user can be detected accurately with a simple configuration.

Further, in the teeth-grinding preventing device of the invention, the sensor portion is arranged at a position opposing the entire mandibular teeth.

In this configuration, the teeth grinding of the user can be detected more accurately.

### Advantageous Effects of Invention

According to the invention, when the teeth grinding is detected, a stimulation is provided to the user and a restraint of the teeth grinding is encouraged even with a small size that can be put into an oral cavity of the user.

### BRIEF DESCRIPTION OF THE DRAWINGS

FIG. 1A is a general explanatory drawing of a teeth grinding preventing device according to an embodiment;

FIG. 1B is an explanatory drawing illustrating a state in which a stimulation providing portion in FIG. 1A is separated from a main body portion;

FIG. 2 is a block diagram illustrating a general configuration of the teeth grinding preventing device of the embodiment;

FIG. 3 is an explanatory cross-sectional view of a sensor portion of the embodiment;

FIG. 4 is an explanatory drawing illustrating a state in which the teeth grinding preventing device of the embodiment is mounted;

FIG. 5 is an enlarged explanatory drawing illustrating a stimulation providing portion in FIG. 1 of the embodiment.

FIG. 6 is a flowchart illustrating an example of a feedback process of the embodiment.

FIG. 7A is a general explanatory drawing of a teeth grinding preventing device according to a modification of the embodiment; and

FIG. 7B is an explanatory drawing illustrating a state in which a stimulation providing portion in FIG. 7A is separated from the main body portion.

### DESCRIPTION OF THE PREFERRED EMBODIMENTS

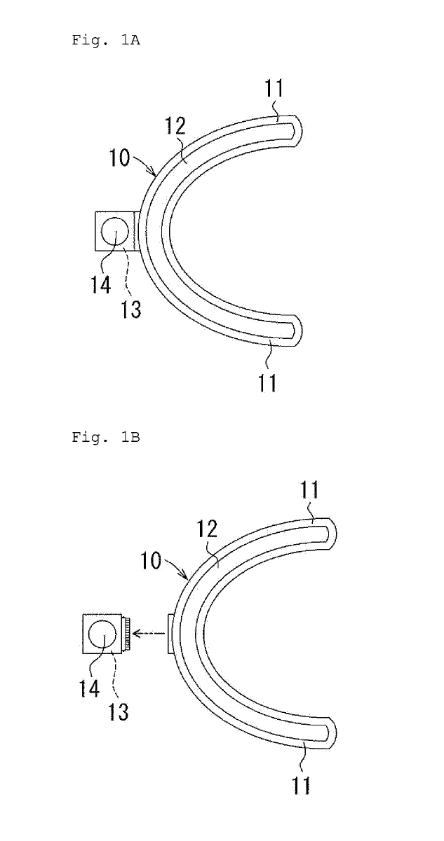

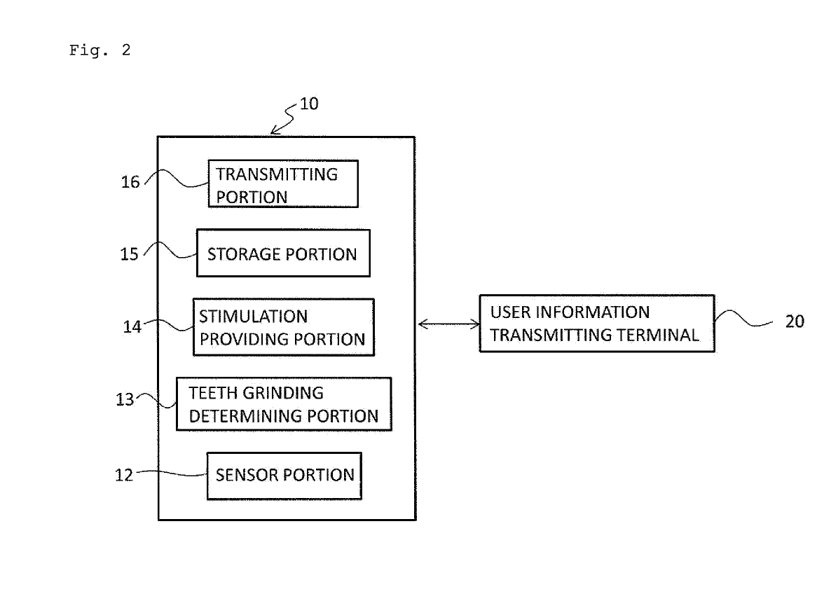

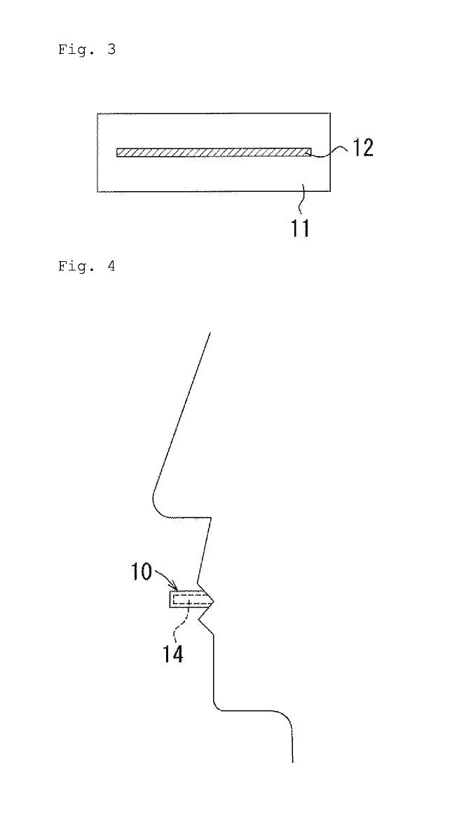

Referring now to FIG. 1 to FIG. 3, a teeth grinding preventing device of an embodiment of the invention will be described. FIG. 1 is a general explanatory drawing of the teeth grinding preventing device of the embodiment. FIG. 2 is a block diagram illustrating a general configuration of the teeth grinding preventing device of the embodiment. FIG. 3 is an explanatory cross-sectional view of a sensor portion of the embodiment.

A teeth grinding preventing device 10 of the embodiment is used by being put in an oral cavity of a user.

The teeth grinding preventing device 10 includes a main body portion 11 formed into a substantially arcuate shape extending along a curve of a maxillary teeth alignment of the user, and configured to cover an occluding surface of the maxillary teeth, a sensor portion 12 embedded in the main body portion 11 so as to extend in parallel to the occluding surface of maxillary and mandibular teeth and configured to detect an occlusal force and output a detection signal, a teeth grinding determining portion 13 configured to receive the detection signal and determine whether or not the user is grinding the teeth, and a stimulation providing portion 14 configured to provide the user with a stimulation when the teeth grinding determining portion 13 determines that the user is grinding the teeth. The stimulation providing portion 14 is provided so as to be mountable and demountable with respect to the main body portion 11.

The main body portion 11 is formed so as to match the maxillary teeth alignment of the user from a denture model of the user, which is formed by a specialist, and is configured to cover an occluding surface of the maxillary teeth. The main body portion 11 is formed of a synthetic resin such as polymethylmethacrylate high-molecular compound, which is widely used in clinical dentistry.

The sensor portion 12 is embedded in the main body portion 11 so as to extend in parallel to the occluding surface of maxillary and mandibular teeth. The sensor portion 12 outputs a detection signal to the teeth grinding determining portion 13 upon detection of the occlusal force. The sensor portion 12 is electrically connected to the teeth grinding determining portion 13 via a line or radio. A configuration including an amplifying portion that amplifies the detected signal is further preferable.

In the embodiment, the sensor portion 12 has a configuration in which a piezoelectric element 12 such as a piezoelectric film or the like is embedded in a substantially arcuate shape, and can detect an occlusal force.

The sensor portion 12 is capable of detecting an accurate occlusal force by being arranged at a position opposing the entire teeth alignment of the mandibular teeth.

The teeth grinding determining portion 13 is configured to receive the detection signal and determine whether or not the user is grinding the teeth. In the embodiment, the teeth grinding determining portion 13 determines that the user is grinding the teeth when the occlusal force with respect to the occluding surface of the maxially and mandibular teeth has a predetermined magnitude or higher from the received detection signal. The predetermined magnitude of the occlusal force is set for each user. In the embodiment, the occlusal force is preferably set to 10% to 20% of a maximum occlusal force that the user being awake can exert spontaneously. The predetermined magnitude of the occlusal force is preferably set to a value corresponding to 15% of the above-described maximum occlusal force. When it is determined that the user is grinding the teeth, the teeth grinding determining portion 13 outputs a teeth grinding detection signal to the stimulation providing portion 14. In contrast, when it is determined that the user is not grinding the teeth, the teeth grinding determining portion 13 does not output a teeth grinding detection signal to the stimulation providing portion 14.

The stimulation providing portion 14 provides the user with stimulation when the teeth grinding determining portion 13 determines that user is grinding the teeth. In other words, the stimulation providing portion 14 provides the user with the stimulation after the stimulation providing portion 14 has received a teeth grinding detection signal. Specifically, the stimulation providing portion 14 provides the user with stimulation, for example, vibrations, sounds, or light. When the user receives some stimulation from the outside while grinding the teeth, the teeth grinding is restrained.

In the embodiment, the stimulation providing portion 14 is composed of a vibrator or the like which provides the user with a vibratory stimulation. The vibratory stimulation contributes to minimize an influence on persons other than the user sleeping in the same room in comparison with sounds or light, and such an event that the sleeping state of the person sleeping in the same room is disturbed may be avoided. Specifically, the stimulation providing portion 14 provides the user with a vibration of a preset amplitude by a preset number of times. The stimulation providing portion 14 is specified to provide the user with 100 times of vibrations per second for 30 seconds by default. However, a configuration in which the amplitude, the number of times of the vibratory stimulation to be provided is changed in accordance with the magnitude of the teeth grinding is also applicable as needed.

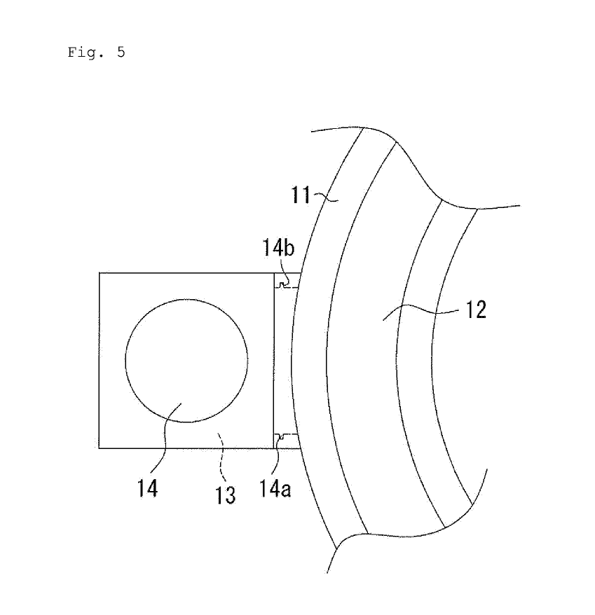

As illustrated in FIG. 4, the stimulation providing portion 14 is provided so as to protrude from a top of the main body portion 11 out of the oral cavity. Since lips follow the shape of the stimulation providing portion 14 easily, sleep of the user is not disturbed.

The stimulation providing portion 14 is provided so as to be mountable and demountable with respect to the main body portion 11. In this embodiment, as illustrated in FIG. 5, the stimulation providing portion 14 includes a pair of projecting portions 14a, and can be coupled with the main body portion 11 by fitting the projecting portions 14a into depressing portions 14b formed on the main body portion 11. For example, when acquiring history-information of the teeth grinding of the user before starting substantial therapy of the teeth grinding, since the stimulation providing portion 14 is mountably and demountably mounted on the main body portion 11, the history information of the teeth grinding of the user can be acquired by demounting the stimulation providing portion 14 without providing the user with a stimulation. When the symptom of the teeth grinding starts to be restrained, a usage as a general mouthpiece is also applicable. In addition, a usage as a splint for the therapy of temporomandibular disorder is also applicable. By demounting the stimulation providing portion 14, light weight of the teeth grinding preventing device 10 can be achieved.

The stimulation providing portion 14 is driven by a drive source such as a compact lithium battery or the like.

The teeth grinding preventing device 10 of the invention preferably includes a storage portion 15 in which time-history data of the occlusal force is stored. The time-history data of the occlusal force corresponds to data indicating a change of the occlusal force with time. The storage portion 15 is composed of a flash memory or the like, and stores the detection signal output from the sensor portion 12, in other words, the time-history data of the occlusal force.

The time-history data of the occlusal force stored in the storage portion 15 can be electrically connected to a laptop personal computer or the like of the user via a connecting code, which is not illustrated, or radio, and the time-history data of the occlusal force can be output to the laptop personal computer of the user, and can be used for analysis of the teeth grinding of the user from the time-history data of the occlusal force. The storage portion 15 may be configured to store sounds caused by the teeth grinding of the user.

Furthermore, the teeth grinding preventing device 10 of the invention may include a transmitting portion 16 configured to transmit the time-history data of the occlusal force stored in the storage portion 15 to a user information communication terminal 20.

In the embodiment, the transmitting portion 16 transmits the time-history data of the teeth grinding stored in the storage portion 15 to the user information communication terminal 20 owned by the user by a near field communication such as Bluetooth (registered trademark). The transmitted time-history data of the teeth grinding may be used for analysis of the teeth grinding of the user, or the like. The user information communication terminal 20 is an information communication terminal owned by the user, and examples thereof include a cellular phone, a smart phone, a tablet terminal, and a laptop personal computer.

With the configuration of the teeth grinding preventing device 10 of the embodiment described above, when the teeth grinding is detected, the teeth grinding can be restrained by providing the user with stimulation such as vibrations.

Here, a number of researchers have disclosed that the teeth grinding of the user is restrained by providing the user with stimulation such as vibration from the outside when the teeth grinding is detected. The inventors of the present application have also confirmed by conducting a clinical examination described below.

Specifically, for the first two weeks, a device having a sensor that detects the teeth grinding was put in an oral cavity for sleep without providing a vibration, and history data of the teeth grinding of a user (woman of 37 years old in this clinical examination) during sleep was recorded. Subsequently, for four months, a vibration was provided to the user when the teeth grinding was detected. Then, the obtained history data of the teeth grinding was analyzed by using a semi-automated custom computer system (stud view SAS). The teeth grinding data shorter than 1 second during a monitoring period was discarded.

As a result of analyzing the history data of the teeth grinding, the number of times when the teeth grinding was detected per hour was reduced by 25% in comparison with that before the therapy after four months have elapsed. The time when the teeth grinding was detected was reduced by 44% in comparison with that before the therapy after four months have elapsed. The user could use the device without specific inconvenience, and substantial sleep disorder caused by putting the device was not reported.

Subsequently, a process sequence to be taken in the case where the teeth grinding preventing device 10 of the invention detects the teeth grinding of the user will be described. FIG. 6 is a flowchart illustrating a process sequence to be taken when the teeth grinding preventing device 10 detects the teeth grinding of the user.

In the case of sleeping, the user puts the teeth grinding preventing device 10 of the invention in the oral cavity. Since the main body portion 11 is formed into a substantially arcuate shape along the curve of the maxillary teeth alignment of the user, the user can put the teeth grinding preventing device 10 without a feeling of discomfort.

In Step S101, when the sensor portion 12 detects an occlusal force by the start of the teeth clenching or the teeth grinding, the sensor portion 12 outputs the detection signal to the teeth grinding determining portion 13. In the embodiment, the sensor portion 12 has a configuration in which a piezoelectric film, for example, is embedded in the substantially arcuate shape, and is capable of detecting an occlusal force with respect to the occluding surface of the maxillary and mandibular teeth. The sensor portion 12 is arranged at a position where the entire mandibular teeth oppose, and an accurate occlusal force can be detected.

In Step S102, the teeth grinding determining portion 13 receives a detection signal output in Step S101, and determines whether or not the user is grinding the teeth. In the embodiment, the teeth grinding determining portion 13 determines that the user is grinding the teeth when the occlusal force has a predetermined magnitude or higher from the received detection signal. The predetermined magnitude of the occlusal force is set for each user. In the embodiment, the occlusal force is preferably set to 10% to 20% of a maximum occlusal force that the user being awake can exert spontaneously, and more preferably, set to 15% of the same. When it is determined that the user is grinding the teeth, the teeth grinding determining portion 13 outputs a teeth grinding detection signal to the stimulation providing portion 14, and the procedure goes to Step S103. In contrast, when the teeth grinding determining portion 13 determines that the user is not grinding the teeth when the occlusal force is smaller than the predetermined magnitude, the procedure goes back to Step S101, and an occlusal force detecting process is continued.

In Step S103, the stimulation providing portion 14 provides the user with stimulation such as vibrations, sounds, or light upon reception of the teeth grinding detection signal output in Step S102. With an application of stimulation from the outside during the teeth grinding, the teeth grinding is restrained.

In the embodiment, the stimulation providing portion 14 provides the user with a vibratory stimulation. Specifically, the stimulation providing portion 14 provides a vibration of a preset amplitude by a preset number of times. The stimulation providing portion 14 is specified to provide 100 times of vibrations per second during 30 seconds by default. The stimulation providing portion 14 is composed of a vibrator or the like which provides the user with a vibratory stimulation. The vibratory stimulation contributes to minimize an influence on persons other than the user sleeping in the same room in comparison with sounds or light, and such an event that the sleeping state of the persons sleeping in the same room is disturbed may be avoided. The stimulation providing portion 14 may also have a configuration in which the amplitude, the number of times, and the like of the vibratory stimulation to be provided are changed in accordance with the magnitude of the teeth grinding.

When the teeth grinding is detected, the teeth grinding preventing device 10 provides the user with a stimulation and can encourage a restraint of the teeth grinding even with a small size that can be put into the oral cavity of the user. Since the teeth grinding preventing device 10 is configured to provide the user with stimulation on the basis of vibrations, sleep of persons other than the user sleeping in the same room is not disturbed. Furthermore, the teeth grinding preventing device 10 is capable of transmitting the time-history data of the teeth grinding of the user to the user information communication terminal owned by the user. Therefore, monitoring of the status quo of the teeth grinding of the user can be monitored.

### Modifications of Embodiment

The invention disclosed in this specification includes a configuration part of which is changed and specified to other configurations of the disclosure in this specification within an applicable range, or a configuration including other configurations of the disclosure in this specification and added thereto and specified, or a configuration from which part of the configuration is deleted within a range that does not impair the advantageous effects and specified as highest conception, in addition to the configuration of the respective modes of the invention and the embodiment, and also includes a modification given below.

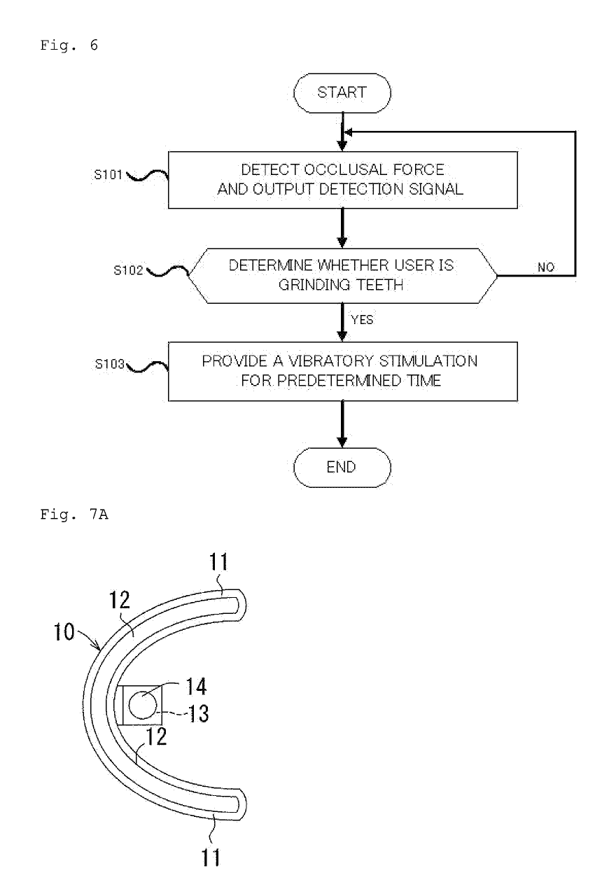

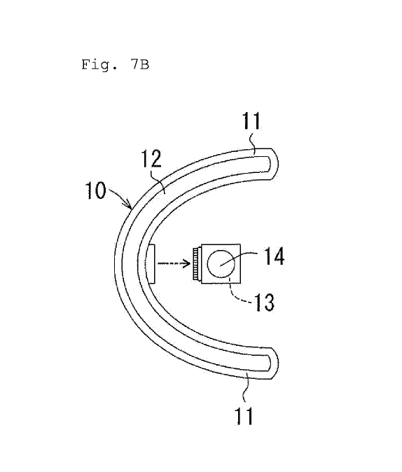

The stimulation providing portion 14 of the embodiment is configured to be provided so as to protrude from the top of the main body portion 11 out of the oral cavity. However, a configuration of being provided from the top of the main body portion 11 within the oral cavity is also applicable as illustrated in FIG. 7.

1. A teeth grinding preventing device to be put in an oral cavity of a user comprising:
a main body portion formed into a substantially arcuate shape along a curve of a maxillary teeth alignment of the user, and configured to cover an occluding surface of the maxillary teeth;
a sensor portion embedded in the main body portion so as to extend in parallel to the occluding surface of maxillary and mandibular teeth, and configured to detect an occlusal force and output a detection signal;
a teeth grinding determining portion configured to receive the detection signal and determine whether or not the user is grinding the teeth; and
a stimulation providing portion configured to provide the user with a stimulation when the teeth grinding determining portion determines that user is grinding the teeth,
wherein the stimulation providing portion is provided so as to be mountable and demountable with respect to the main body portion.

2. The teeth grinding preventing device according to claim 1, further comprising a storage portion in which time-history data of the occlusal force is stored.

3. The teeth grinding preventing device according to claim 2, further comprising a transmitting portion configured to transmit the time-history data of the occlusal force stored in the storage portion to a user information communication terminal.

4. The teeth grinding preventing device according to claim 1, wherein the stimulation providing portion provides the user with a vibratory stimulation.

5. The teeth grinding preventing device according to claim 1, wherein the stimulation providing portion is provided so as to protrude from a top of the main body portion out of the oral cavity.

6. The teeth grinding preventing device according to claim 1, wherein the sensor portion is composed of a piezoelectric element embedded in a substantially arcuate shape.

7. The teeth grinding preventing device according to claim 1, wherein the sensor portion is arranged at a position opposing the entire mandibular teeth.

8. The teeth grinding preventing device according to claim 2, wherein the stimulation providing portion provides the user with a vibratory stimulation.

9. The teeth grinding preventing device according to claim 3, wherein the stimulation providing portion provides the user with a vibratory stimulation.

10. The teeth grinding preventing device according to claim 2, wherein the stimulation providing portion is provided so as to protrude from a top of the main body portion out of the oral cavity.

11. The teeth grinding preventing device according to claim 3, wherein the stimulation providing portion is provided so as to protrude from a top of the main body portion out of the oral cavity.

12. The teeth grinding preventing device according to claim 4, wherein the stimulation providing portion is provided so as to protrude from a top of the main body portion out of the oral cavity.

13. The teeth grinding preventing device according to claim 2, wherein the sensor portion is composed of a piezoelectric element embedded in a substantially arcuate shape.

14. The teeth grinding preventing device according to claim 3, wherein the sensor portion is composed of a piezoelectric element embedded in a substantially arcuate shape.

15. The teeth grinding preventing device according to claim 4, wherein the sensor portion is composed of a piezoelectric element embedded in a substantially arcuate shape.

16. The teeth grinding preventing device according to claim 5, wherein the sensor portion is composed of a piezoelectric element embedded in a substantially arcuate shape.

17. The teeth grinding preventing device according to claim 2, wherein the sensor portion is arranged at a position opposing the entire mandibular teeth.

18. The teeth grinding preventing device according to claim 3, wherein the sensor portion is arranged at a position opposing the entire mandibular teeth.

19. The teeth grinding preventing device according to claim 4, wherein the sensor portion is arranged at a position opposing the entire mandibular teeth.

20. The teeth grinding preventing device according to claim 5, wherein the sensor portion is arranged at a position opposing the entire mandibular teeth.
